(19) United States
(12) Patent Application Publication
(10) Pub. No.: US 2010/0129268 A1
(43) Pub. Date: May 27, 2010

(54) AROMA PROJECTING DEVICE AND METHOD

(75) Inventor: Jacob Ranis Stokholm Andersen, Hong Kong (CN)

Correspondence Address: VOLPE AND KOENIG, P.C., UNITED PLAZA, SUITE 1600, 30 SOUTH 17TH STREET, PHILADELPHIA, PA, 19103, US

(73) Assignee: HOME FOCUS DEVELOPMENT LTD., Tortola (VG)

(21) Appl. No.: 12/063,157

(22) PCT Filed: Aug. 8, 2006
(86) PCT No.: PCT/IB2006/002178

§ 371 Date: Feb. 7, 2008

Related U.S. Application Data
(60) Provisional application No. 60/706,461, filed on Aug. 8, 2005.

(51) Int. Cl. A62B 7/08; A61L 9/00; F02M 17/16

(52) U.S. Cl. 422/124; 705/500; 53/473; 422/4; 422/5; 261/88

(57) ABSTRACT

The invention relates to a device (1), particularly a child-safe device, for projecting and or directing a scent that includes a housing (10) having a drive mechanism (30) contained therein, a housing cap (20) to enclose the housing (10), an air projector removably connected to a drive shaft of the drive mechanism (30), and a scent-releasing substance in the vicinity of the air projector; as well as methods of using, refilling and marketing the device.

### FIELD OF THE INVENTION

The invention relates to the field of scent dispersing, more particularly to a device for projecting and directing a scent.

### BACKGROUND

The use of scents for improving the smell of one's environment is well known in the art, for example scented candles and air fresheners. Such air fresheners may be either portable or may be limited for use with an electrical outlet. Typically, such methods use diffusion principles, forcible ejection methods via propellants, or a combination of the two methods for transferring a particular scent from its container into the environment. The environment into which the scent is dispersed is typically one having a significant cubic volume of air, such as a room or automobile or even outdoors. However, such methods are problematic for several reasons. For example, a particular scent may be agreeable to some and disagreeable to others, yet there are no simple and effective methods of limiting others' exposure to the scent. Also, the intermittent use for which many conventional devices are designed or those relying only on diffusion result in a temporary scent cloud. If used outdoors, particularly when one is walking or otherwise moving, the scent cloud quickly disseminates or is walked-through such that the beneficial effects of the scent are quickly minimized or eliminated. Furthermore, if a user forcibly ejects the scent (along with potentially harmful components such as propellants) in close proximity to the face, there is the likelihood that skin and/or eye irritation would result. Finally, conventional air fresheners typically dispense large amounts of scent in large cubic volume environments, where other environmental scents are to be covered or masked, thereby overwhelming not only any other scent in the environment but also the olfactory nerves of the user.

Thus, there is a need within the art for a device capable of projecting and directing a scent that is substantially perceived only by the user.

### SUMMARY OF THE INVENTION

The invention generally relates to a device for projecting scent comprising (a) a housing having an interior space; (b) a housing cap, wherein the cap substantially encloses the housing; (c) a drive mechanism, wherein the drive mechanism is contained within the housing; (d) an air projector removably connected to the drive mechanism; and (e) a scent-releasing substance in the vicinity of the air projector.

The invention also relates to a device for projecting scent comprising (a) a housing having an interior space; (b) a housing cap, wherein the cap substantially encloses the housing; (c) a drive mechanism comprising a drive shaft; a motor rotatably connected to the drive shaft, a power source, and a plurality of leads connecting the power source to the motor, wherein the drive mechanism is contained within the housing; (d) a fan assembly removably connected to the drive shaft, wherein the fan assembly comprises a plurality of blades, a scent reservoir having the plurality of blades disposed thereon, a rotatable cover disposed over the scent reservoir, the rotatable cover having at least one aperture therethrough; and (e) a scent-releasing substance contained in the scent reservoir.

The invention also relates to a scent delivery device comprising (a) a housing having an interior space; (b) a housing cap, wherein the cap substantially encloses the housing; (c) a drive mechanism comprising a drive shaft; a motor rotatably connected to the drive shaft, a power source, and a plurality of leads connecting the power source to the motor, wherein the drive mechanism is contained within the housing; (d) a fan assembly removably connected to the drive shaft, wherein the fan assembly comprises a plurality of blades, a scent reservoir having the plurality of blades disposed thereon, a rotatable cover disposed over the scent reservoir, the rotatable cover having at least one aperture therethrough; and (e) a scent-releasing substance contained in the scent reservoir, wherein the device is configured to be child-safe.

The invention also relates to a method of directing and projecting a scent comprising providing a device of the invention, projecting air with an air projector containing a scent-releasing substance; and pointing the fan assembly in the proximity of a user's face.

The invention also relates to a method of refilling a scent-releasing substance of the device comprising removing a scent-releasing substance from the device; and inserting new scent-releasing substance.

The invention also relates to a method of marketing a scent directing and projecting device comprising packaging the scent projecting device in combination with a confectionary product; selling the combination package with at least one scent-pack refill; and instructing a user to control the level of scent emission, optimal proximity to the user's face and removing/replacing the fan assembly or secondary structure.

### BRIEF DESCRIPTION OF THE DRAWINGS

For the purpose of illustrating the devices of the invention, there is shown in the drawings a form which is presently preferred; it being understood, that this invention is not limited to the precise arrangements and instrumentalities shown.

FIG. 1 is a side view of an exemplary device of the invention.

FIG. 2 is a cross-sectional side view of an exemplary device of the invention.

FIG. 3 is a frontal view of an exemplary device of the invention.

FIG. 4 is a frontal view of an exemplary device of the invention.

FIG. 5 is a side view of an exemplary device of the invention.

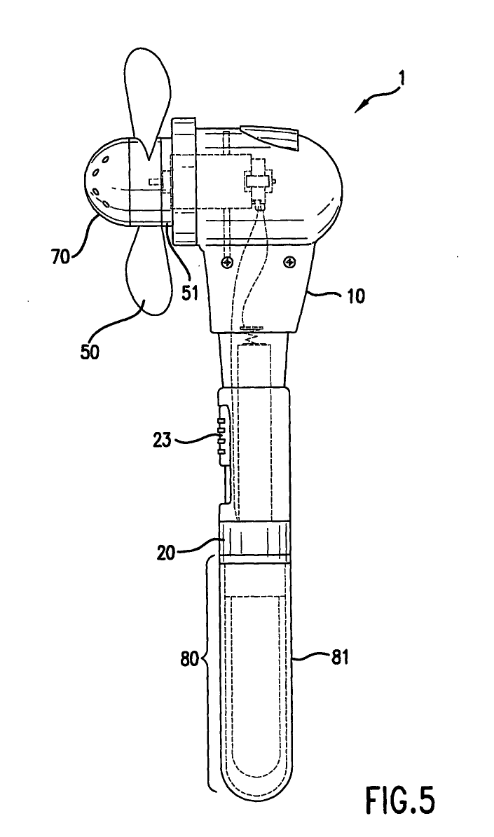

FIG. 6 is a cross-sectional view showing a drive mechanism of an exemplary device of the invention.

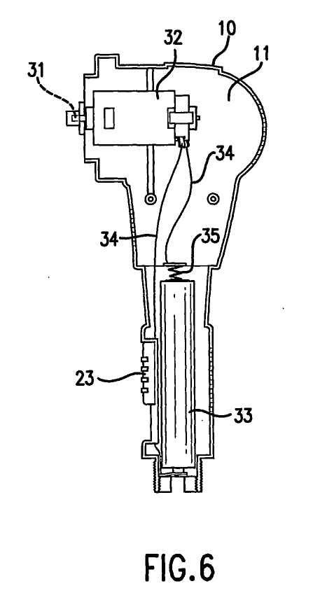

FIG. 7 is a frontal view of an exemplary device of the invention.

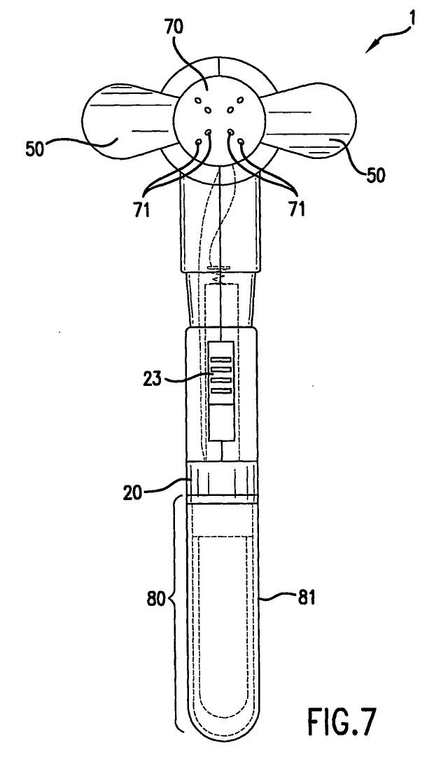

FIG. 8 is a frontal view of an exemplary device of the invention.

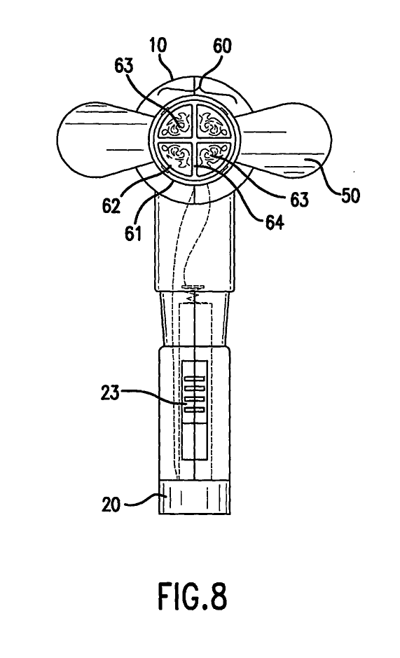

### DETAILED DESCRIPTION

It will be appreciated that the following description is intended to refer to specific devices of the invention selected for illustration in the drawings and is not intended to define or limit the invention, other than in the appended claims.

As used herein, the term “scent” and any derivations thereof refer to an element, ingredient, chemical, composition and/or substance that acts as a stimulant to olfactory sensory receptors, and specifically includes any fragrance, and/or aroma, and/or odor, and/or smell, whether pleasing or offensive. It is understood that “scent” includes the use of a single scent or the combination of two or more scents either in a mixture or individually.

As used herein, the term “local environment” refers to the area that is substantially immediate to the device of the invention such as, for example, a radius of about five feet; for example, about four feet, about three feet, about two feet or about one foot, or about six inches or less. The local environment may encompass not only the operator, but also a recipient towards whom the device and scent are directed (collectively referred to as a “user”).

The invention provides a device for projecting a scent, wherein scent is emitted from the device and propelled into the local environment. The devices of the invention enhance a user's enjoyment of their local environment. The device offers directed on-demand delivery of a scent to a user, where the scent is substantially perceived and enjoyed only by the user in a given environment, thereby limiting others' exposure to the scent.

The device can be used outdoors and can deliver a scent to the user whenever the user activates the device. Thus, the user can project the scent into the local environment and can perceive the scent when desired without experiencing dissipation of or walking through the scent cloud. Thus, the scent does not overwhelm other scents in the environment.

Projection of the scent does not require extraneous propulsive chemicals such as, for example, propellants that may be harmful to both the user and the environment. Instead, devices of the invention can utilize a non-pressurized volatile scent, a pump mechanism to spritz or spray a scent in the vicinity of the air projector or any other method known to one skilled in the art.

Additionally, the devices of the invention can be child-safe, such that their structural and chemical components are non-toxic, and safe for use by small children such as, for example about 3 years of age and older. For example, the device of the invention can be in conformity with standard child safety protocols or guidelines, such as European Standard BS EN 71 or its US equivalent.

Further, the devices of the invention can be used or distributed in combination with a secondary structure such as a confectionary product and, as a result, a user may experience both olfactory and taste sensations, thereby even further enhancing their experience and enjoyment.

Generally, the devices of the invention do not require the application of heat to disperse, emit or give-off the desired scent. However, the device can comprise a mild heating element known to those skilled in the art, for example, one powered by the power source, contained within the device as described more fully below. When scent-releasing substances which release a scent upon the application of heat are used, such as certain gel matrices known in the art, the heat enhances the dispersion of the scent contained therein.

Generally, the device for projecting a scent comprises (a) a housing having an interior space; (b) a housing cap, wherein the cap substantially encloses the housing; (c) a drive mechanism, wherein the drive mechanism is contained within the housing; (d) an air projector removably connected to the drive mechanism; and (e) a scent-releasing substance in the vicinity of the air projector, where the scent-releasing substance is positioned relative to the air projector so that the projected air disperses scent. Typically, the scent-releasing substance is contained within the air projector.

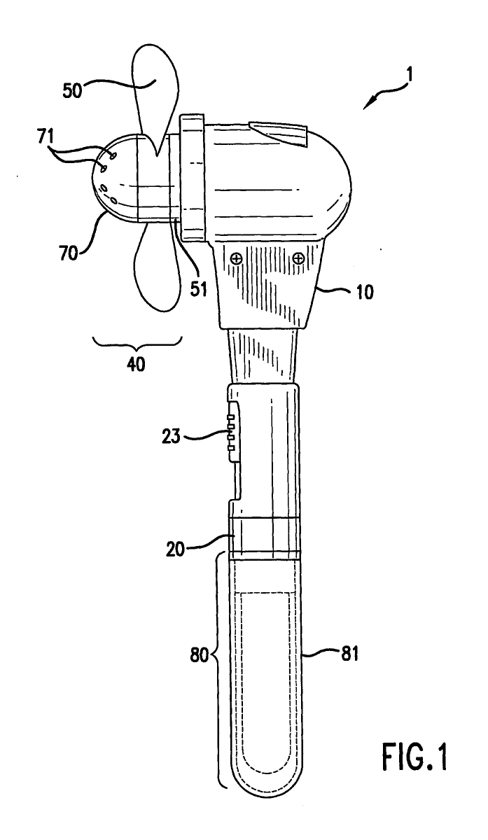

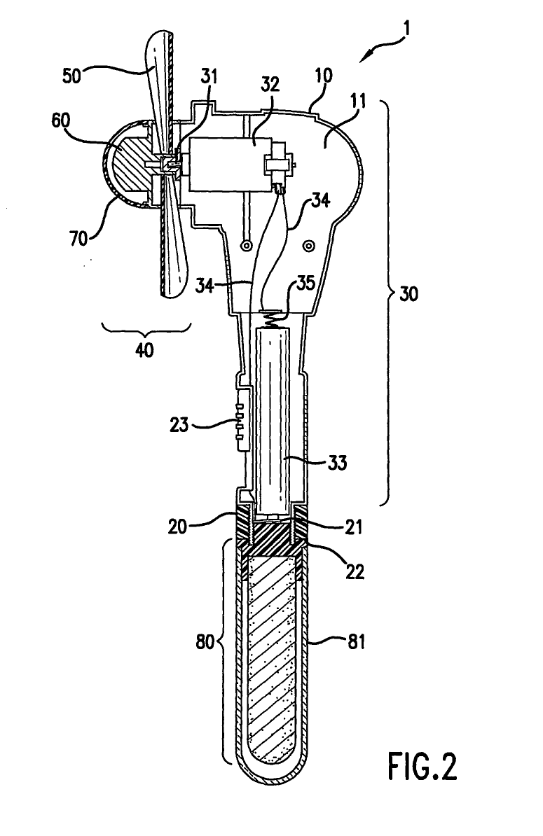

In the figures, in which like reference numerals indicate like elements, there is shown an exemplary scent projection device. With reference to FIGS. 1, 2 and 3, the device 1 typically comprises (a) a housing 10 having an interior space 11; (b) a housing cap 20, wherein the cap 20 substantially encloses the housing 10; (c) a drive mechanism 30 comprising a drive shaft 31; a motor 32 rotatably connected to the drive shaft 31, a power source 33, and a plurality of leads 34 connecting the power source 33 to the motor 32, wherein the drive mechanism 30 is contained within the housing 10; and (d) a fan assembly 40 removably connected to the drive shaft 31, wherein the fan assembly 40 comprises a plurality of blades 50, a scent reservoir 60 having the plurality of blades 50 disposed thereon, a rotatable cover 70 disposed over the scent reservoir 60, the rotatable cover 70 having at least one aperture 71 therethrough.

The housing 10 may comprise any suitable shape capable of accommodating the drive mechanism 30 as well as other components of the device 1 such as, for example, the housing cap 20, scent reservoir 60, and air projector. The housing 10, drive mechanism 30, air projector, scent reservoir 60 and any other components may be scaled to either larger or smaller sizes as appropriate as would be readily recognized and known by one skilled in the art. Exemplary suitable shapes for the housing 10 include humans, animals, generic geometric shapes, corporate logos, toy shapes (e.g. cars, planes, trains, and robots) or fanciful shapes. Moreover, the housing 10 may comprise one or more colors, designs or other fanciful indicia, and any combination thereof, which may be integrated into the shape of the housing or may be applied post-manufacture in many forms, including stickers, decals, appliqués and various combinations thereof.

The housing 10 may be constructed of any suitably rigid material such as, for example, plastics, metals, rubbers, synthetic resins, cardboard, wood or any combination thereof, wherein the use of such materials is well known by those skilled in the art. Preferably, the housing 10 is constructed of plastic. The housing 10 may be produced by any method generally known in the art suitable for use with a given material, such as, for example, injection molding for plastic.

The housing 10 is generally hollow, thereby having a defined interior 11 throughout at least a portion of its structure. While portions of the housing 10 may be solid, an entirely hollow structure can be used. The housing 10 also typically provides a method for instituting electrical contact between the power source 33 and the motor 32 such as, for example, an ON/OFF switch 23.

The housing cap 20 for use in conjunction with the housing 10 may have any configuration necessary and/or known by those skilled in the art to enclose the housing 10. The cap 20 may be threaded, ribbed or have some other configuration to allow it to snap onto the housing 10, thereby securing any contents therein. The cap 20 may further comprise an electrically conductive piece (e.g. a metal piece) disposed on an interior surface 21 of the cap 20 (the surface of the cap facing the interior of the housing) for use as a contact in combination with the ON/OFF switch 23 and the power source 33, thereby forming a circuit to provide power to the motor 32. The cap 20 may further serve as a method of detachably connecting a secondary structure 80 to the housing 10, where both the interior 21 and exterior 22 (the surface facing away from the housing's interior) cap surfaces may be threaded, ribbed or have some other configuration to allow it to snap onto the housing 10 to secure the contents therein. The housing cap 20 thus can permit the secondary structure 80 to be readily engaged and disengaged from the housing 10 for independent use apart from the devices of the invention or for use in combination with the devices of the invention.

The drive mechanism can comprise any mechanism for powering the air projector. Such mechanisms can include a gear arrangement, a pump/trigger arrangement, a solar power arrangement and any other method known to one skilled in the art. Typically, the drive mechanism 30 comprises a drive shaft 31; a motor 32 rotatably connected to the drive shaft 31, a power source 33, and a plurality of leads 34 electrically connecting the power source 33 to the motor 34. The power source 33 may be any type known to those skilled in the art, including a battery, for example a AA battery. The leads 34 are typically wire as is known to those skilled in the art, for example copper or aluminum wire, and include an electrical contact 35. The motor 32 may be any appropriate electric motor known and available to those skilled in the art. The drive shaft 31 rotates the fan assembly 40 in order to project the scent. The drive shaft 31 extends from the motor 32 through the housing 10 and allows for removeable attachment of the fan assembly 40. Suitable methods for ensuring removable attachment are known to those skilled in the art. The drive shaft 31 may be constructed of any suitable material such as, for example, plastics, metals or other material known to those skilled in the art.

The invention may utilize any air projector suitable for projecting a scent known to one skilled in the art, but generally uses a fan assembly 40. The fan assembly 40 is removably attached to the drive shaft 31 such that the user may detach it from the drive shaft 31. The fan assembly 40 can comprise a plurality of blades 50, a scent reservoir 60 having the plurality of blades 50 disposed thereon, a rotatable cover 70 disposed over the scent reservoir 60, the rotatable cover 70 having at least one aperture 71 therethrough. The fan assembly 40 uses the plurality of blades 50 to draw air past the scent reservoir 60 and scent-releasing substance, thereby projecting and directing the scent.

The plurality of blades 50 are typically those having the configuration of a conventional fan blade, wherein the blades 50 extend radially away from the rotational axis of the fan assembly 40 and, upon rotating, the blades 50 are able to direct and project air as well as any scent dispersed therein. The blades 50 may be constructed of any suitable material known to those skilled in the art such as, for example, metal, plastic, foam or a combination of these materials. Generally, the plurality of blades are comprised of a pliable soft foam comprising a polymer such as, for example, ethylene vinyl acetate (EVA). The blades 50 may be formed according to those methods known in the art for a given material, such as injection molding in the case of EVA. Typically, there are two blades, however 3 or 4 or more blades can be used.

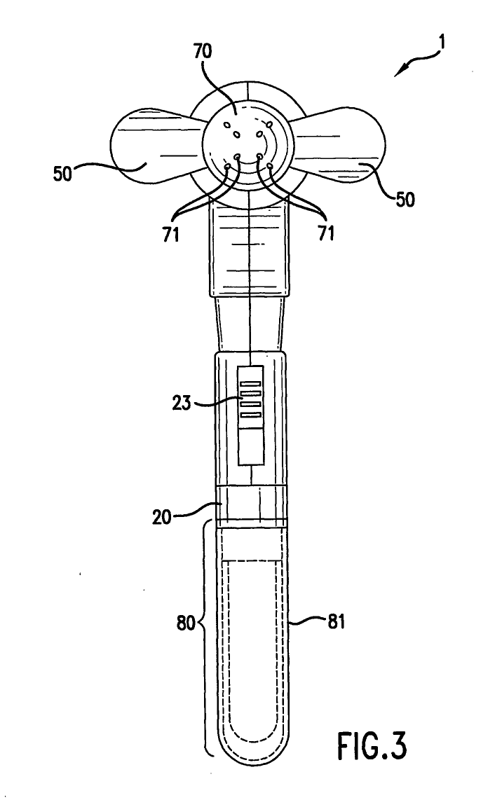

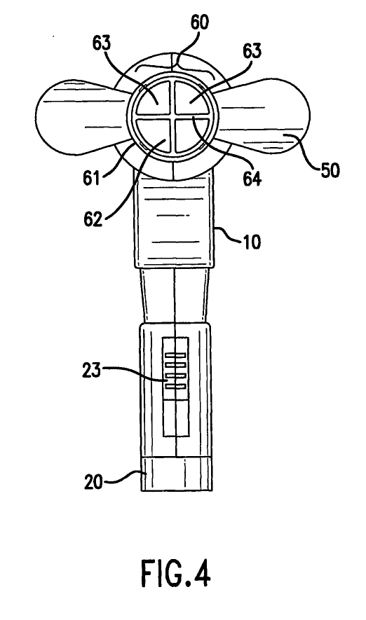

The scent reservoir can be positioned in a number of places and can comprise many different configurations as known by one skilled in the art. For example, the scent reservoir can be a container and may be located in the interior of the housing, where it is in fluid connection with the environment by using a tube. With reference to FIGS. 3 and 4, the scent reservoir 60 comprises a base 61 and at least one chamber 62, cavity or well or other concave configuration (e.g. bowl-shaped) that contains a scent-releasing substance. The base 61 is posterior to the at least one chamber 62 and typically form a unitary structure. The base 61 allows for the proper positioning of the plurality of blades 50 that are disposed on the back thereof while also providing for removeable attachment of the rotatable cover 70. The plurality of blades 50 may be locked into portion using a backing 51. The backing 51 is typically complimentary in shape to the base 61 of the scent reservoir 60. The backing 51 can be posteriorly engaged with the base 61 thereby ensuring proper positioning such as the blade angle. Both the methods of positioning the blades 50 and removable attachment of the rotatable cover 70 to the base 61 are well known to those skilled in the art. The at least one chamber 62 is defined by a side wall and floor and can comprise four sub-chambers 63 completely separated from one another by cross-walls 64 such that one or more scents can be used simultaneously without substantial direct contact among the various scent-releasing substances.

The rotatable cover 70 comprises at least one aperture 71 therethrough, and provides for containment of the scent-releasing substance while also allowing for the controlled release of the scent. The rotatable cover 70 may have any suitable shape known to those skilled in the art, however it is preferably concave or dome-shaped. The rotatable cover 70 engages with the base 61 of the scent reservoir 60 such that a user may rotate the cover 70 where the at least one aperture 71 is open and allows for the passage of the scent. Conversely, the user may counter-rotate the cover 70 to close the at least one aperture 71 thereby substantially preventing release of the scent. As a result, when not in use, the device of the invention allows for the preservation of the scent-releasing substance, which further extends the useful life of scent-releasing substance. The rotatable cover 70 may be engaged with the base 61 of the scent reservoir 60 using those methods known to one skilled in the art. Thus, certain devices of the invention not only provide a scent to a user, but, by allowing for controlled scent release, the user can use the devices of the invention to project air for its cooling effects without substantial projection or dispersion of the scent.

The fan assembly 40 can be removably attached or connected to the drive shaft of the drive mechanism 30, whereby the fan assembly 40 may be easily disengaged or detached. Those skilled in the art readily understand the methods by which such a removeable attachment or connection is effectuated. As a result, the devices of the invention can further comprise a secondary structure 80 that can be engaged with the drive shaft 31 and enjoyed either independently or in combination with any scent.

The scent reservoir 60, backing 51 and rotatable cover 70 may be constructed of any suitably rigid material such as, for example, plastics, metals, rubbers, synthetic resins, cardboard, wood or any combination thereof, wherein the use of such materials is well known by those skilled in the art. Generally, the scent reservoir 60 and rotatable cover 70 are constructed of plastic. The scent reservoir 60 and rotatable cover 70 may be produced by any method generally known in the art for a given material; for example, injection molding for plastic.

The scent can be formulated as a component or ingredient of a scent-releasing substance. The scent-releasing substance may be in the form of a solid, gel matrix or liquid (e.g., an oil) or other scent-producing/releasing form (e.g., a semi-solid such as a wax) known in the art that allows for the evaporation, vaporization, dispersing, exuding, emitting or giving-off of a scent. The processes for making such scent-release substances are known by those skilled in the art. For example, gel formulations can comprise a gel matrix derived from an organic or inorganic system and are typically prepared hot, and the scent is added as the product cools to preserve the integrity of the scent. Preferably the scent-releasing substance is capable of releasing a scent for a prolonged or extended period of time such as, for example, at least about one day, at least about a week, or at least about a month.

Any scent known in the art may be used with the devices of the invention. Formulations for such scents along with the processes for making them are known in the art. The scent may be of natural origins (e.g., made with plant, flower or fruit extracts), chemically synthesized according to processes known to those skilled in the art, or a combination of natural and synthetic scents. Generally, the scent for use in the invention is non-toxic and child-safe. The scent-releasing substance can utilize a scent in a concentrated form, so as to minimize the volume used and to maximize the time such scent-releasing substances are capable of providing their scent.

Representative examples of scent-producing chemicals include aldehydes. When concentrated, aldehydes smell soapy or fatty; however, when mixed in the proper proportions with water, they develop a sweet, flowery smell. “Green” scents are fresh, having an odor similar to cut grass or plant stems, and are also generally produced by synthetic perfume oils. However, natural sources of green notes such as galbanum (a tropical resin) or violet leaf oil can also be used. “Floral” notes are some of the oldest and most popular scent components. Examples include jasmine-rose complexes blended with lilac and lily-of-the-valley. “Herbal-spice” scent notes can also be used. Lavender, sage, moss, cinnamon, cloves, sandalwood, and cedar can be used to provide these notes. “Oriental” scent notes can be used. Oriental scent notes are sweet, heavy, and strong, and are often found in natural animal materials, such as musk.

Other examples of suitable scent ingredients for use in the invention include bergamot, bitter orange, lemon, mandarin, caraway, cedar leaf, cloverleaf, cedarwood, geranium, lavender, patchouli, lavandan, rose absolute, geranyl (apple), citral (lemon), bornyl isovalerate (pine) and ethylmethylphenyl glycidate (strawberry), and the like, or combinations thereof. These scent-producing chemicals can be mixed with a variety of synthetic scent components, such as aldehydes, ketones, esters, alcohol, terpenes, and the like, or combinations thereof.

Exemplary scents include, but are not limited to, candy (e.g., bubblegum, cotton candy, caramel apple, popcorn, and chocolate), fruit scents (e.g., grape, lemon, orange, cherry, lime, apple, strawberry and watermelon), cola, floral scents, the ocean, herbs, pine and a variety of food items (e.g., peanut butter, pizza, hot dogs, and hamburgers). Additionally, the scent used in the device of the invention need not be pleasurable, but instead may be unusual, displeasing or offensive. Such scents include garbage, vomit, limburger cheese, onions or garlic. It is contemplated that combinations of scents can be used such that innumerable scent combinations are available to the user.

Typically, when the scent is a component of a volatile liquid, it is retained in an absorbent material of types including cotton or other cellulose based material, flexible foam or other absorbent or sponge-like material capable of retaining the scent-releasing substance known to those skilled in the art, for example as described in U.S. Pat. No. 4,283,011 which is herein incorporated by reference in its entirety. When the scent is used in the form of a gel matrix, the use of an absorbent material is optional. Those skilled in the art recognize and understand the appropriate components of a gel matrix useful in the invention and the process used for producing the scent-releasing gel matrices, for example as described in U.S. Patent Publication 2004/0188535 which is herein incorporated by reference in its entirety. Similarly, those skilled in the art recognize and understand the methods and process for forming the scent-releasing liquids, solids and semi-solids that may be used with the devices of the invention. The scent-releasing substances are readily refillable, such that once the scent has been substantially depleted, the scent reservoir may be refilled with either the same or different scent-releasing substance and the same or different scent. Generally, the scent-releasing substances described herein are non-reactive with the other components and materials used in the device of the invention.

The air projector may be any suitable air projector known to those skilled in the art, such as a fan assembly. As noted above, the fan assembly 40 can be removably attached or connected to the drive shaft 31 of the drive mechanism 30, whereby the fan assembly 40 may be disengaged and a secondary structure 80 may be engaged with the drive shaft 31. Also as noted above, the secondary structure 80 may be readily engaged and disengaged from the housing cap 20, thereby permitting independent use of the secondary structure 80 or use in combination with the device of the invention by removably attaching it to the drive shaft 31. The secondary structure 80 can comprise a confectionary product 90 (e.g., a candy product such as lollipops, stick candy or other types of hard candy known to those skilled in the art), wherein the device of the invention can rotate the secondary structure 80, thereby enhancing both the pace of enjoyment of consumption for the user. The secondary structure 80 may originate from outside sources independent of the device of the invention (e.g., separate scent-pack refills) or as noted above, may be detachable from the exterior 22 face of the cap 20 and placed in rotatable connection with the drive shaft 31 and subsequently replaced onto the cap's exterior face 22. Moreover, a secondary structure cap 81 may be used to cover the structure. For example when candy is the secondary structure 80, the cover prevents contact with foreign substances or contamination from dirt or other elements that may affect the taste of the candy.

In those instances where the secondary structure 80 is candy, it may be enjoyed by itself or it may be complimentary to the scent contained in the scent reservoir 60 such that perception of the scent and consumption of the candy provide a combined sensory effect to the user. Exemplary combinations include peanut butter and jelly, applies and cinnamon, lemon and lime, chocolate and vanilla, strawberry and banana, cherry and vanilla, or suitable or well-known combination of tastes and scents desired to be experienced by a user.

To assist in properly operating the device of the invention, instructions can be provided with the device that provide information regarding operation and enjoyment of the device, including the control of scent dispersing, optimal proximity to the user's face, and removing/replacing the air projector (e.g., fan assembly) or secondary structure.

The invention also provides to a method of marketing a scent projecting device. The marketing method involves packaging the scent projecting device of the invention in combination with a confectionary product as shown in FIG. 1, 2 or 3. The combination package can then be sold along with a variety of scent-packs (which can comprise packages containing refills of any of the variety of scent-releasing substances).

The device of the invention can be further marketed with other items such as electronic devices (such as for keeping time or producing light and/or sound effects) and promotional items such as contests, lotteries, stickers, collectible items (e.g., cards and toys).

A variety of modifications to the devices described will be apparent to those skilled in the art from the disclosure provided herein. Thus, the invention may be embodied in other specific forms without departing from the spirit or essential attributes thereof and, accordingly, reference should be made to the appended claims, rather than to the foregoing specification, as indicating the scope of the invention.

What is claimed is:

1. A device for projecting a scent comprising:
(a) a housing having an interior space;
(b) a housing cap, wherein the cap substantially encloses the housing;
(c) a drive mechanism, wherein the drive mechanism is contained within the housing;
(d) an air projector removably connected to the drive mechanism; and
(e) a scent-releasing substance in the vicinity of the air projector.

2. The device according to claim 1, further comprising:
(f) a secondary structure removably attached to the housing cap.

3. The device according to claim 1, wherein the drive mechanism comprises a drive shaft; a motor rotatably connected to the drive shaft, a power source, and a plurality of leads connecting the power source to the motor.

4. The device according to claim 1, wherein the air projector is a fan assembly removably connected to the drive shaft, wherein the fan assembly comprises a plurality of blades, a scent reservoir having the plurality of blades disposed thereon, a rotatable cover disposed over the scent reservoir, the rotatable cover having at least one aperture therethrough.

5. The device according to claim 1, wherein the scent-releasing substance is contained in the housing.

6. The device according to claim 4, wherein the scent-releasing substance is contained in the scent reservoir.

7. The device according to claim 1, wherein the air projector is contained within the housing.

8. The device according to claim 1, wherein the air projector is outside the housing.

9. The device according to claim 1, wherein the scent-releasing substance is contained in the air projector.

10. A device for projecting a scent comprising:
(a) a housing having an interior space;
(b) a housing cap, wherein the cap substantially encloses the housing;
(c) a drive mechanism comprising a drive shaft; a motor rotatably connected to the drive shaft, a power source, and a plurality of leads connecting the power source to the motor, wherein the drive mechanism is contained within the housing;
(d) a fan assembly removably connected to the drive shaft, wherein the fan assembly comprises a plurality of blades, a scent reservoir having the plurality of blades disposed thereon, a rotatable cover disposed over the scent reservoir, the rotatable cover having at least one aperture therethrough; and
(e) a scent-releasing substance contained in the scent reservoir.

11. The device according to claim 10, wherein the housing is in the form a human, animal, geometric shape, logo, car, plane, train, robot or fanciful shape.

12. The device according to claim 10, wherein the housing cap further comprises an interior face and an exterior face.

13. The device according to claim 12, wherein the interior face has an electrically conductive piece disposed thereon.

14. The device according to claim 10, wherein the power source is a battery.

15. The device according to claim 14, wherein the battery is a AA battery.

16. The device according to claim 10, wherein the plurality of blades comprises a pliable foam.

17. The device according to claim 16, wherein the pliable foam comprises a polymer.

18. The device according to claim 17, wherein the polymer is ethylene vinyl acetate.

19. The device according to claim 10, wherein the plurality of blades are child-safe.

20. The device according to claim 10, wherein the scent reservoir further comprises a base and at least one chamber, wherein the base and at least one chamber form a unitary structure.

21. The device according to claim 20, wherein the at least one chamber comprises four sub-chambers completely separated by cross-walls.

22. The device according to claim 20, wherein the fan assembly further comprises a backing posteriorly engaged with the base of the scent reservoir, wherein the backing has a shape that is complimentary to the base of the scent reservoir.

23. The device according to claim 10, wherein the scent-releasing substance is a liquid, gel matrix, semi-solid or solid.

24. The device according to claim 10, wherein scent is of natural origin.

25. The device according to claim 10, wherein the scent is a chemically synthesized scent.

26. The device according to claim 10, further comprising.
(f) a secondary structure removably attached to the housing cap.

27. The device according to claim 26, wherein the secondary structure is a confectionary product.

28. A scent delivery device comprising:
(a) a housing having an interior space;
(b) a housing cap, wherein the cap substantially encloses the housing;
(c) a drive mechanism comprising a drive shaft; a motor rotatably connected to the drive shaft, a power source, and a plurality of leads connecting the power source to the motor, wherein the drive mechanism is contained within the housing;
(d) a fan assembly removably connected to the drive shaft, wherein the fan assembly comprises a plurality of blades, a scent reservoir having the plurality of blades disposed thereon, a rotatable cover disposed over the scent reservoir, the rotatable cover having at least one aperture therethrough; and
(e) a scent-releasing substance contained in the scent reservoir;
wherein the device is configured to be child-safe.

29. The device according to claim 28, further comprising:
(f) a secondary structure removably attached to the housing cap.

30. The device according to claim 28, wherein the plurality of blades comprises ethylene vinyl acetate.

31. A method of directing and projecting a scent comprising:
1. providing the device of claim 1;
2. projecting air with an air projector containing a scent-releasing substance; and
3. pointing the air projector towards a user's face, wherein the scent is substantially perceived only by the user.

32. A method of refilling a scent-releasing substance of the device according to claim 1 comprising:
A. removing a scent-releasing substance from the device; and
B. inserting a new scent-releasing substance.

33. A method of marketing a scent directing and projecting device according to claim 1 comprising:
packaging the scent projecting device in combination with a confectionary product;
selling the combination package with at least one scent-pack refill; and
instructing a user to control the level of scent emission, optimal proximity to the user's face and removing/replacing a fan assembly or a secondary structure.
